(19) United States
(12) Patent Application Publication
(10) Pub. No.: US 2025/0134642 A1
(43) Pub. Date: May 1, 2025

(54) Vascular Implant

(71) Applicant: BARD PERIPHERAL VASCULAR, INC., Franklin Lakes, NJ (US)

(72) Inventors: Matt Casiraro, Tempe, AZ (US); Alexander Lastovich, Gilbert, AZ (US)

(73) Assignee: BARD PERIPHERAL VASCULAR, INC., Franklin Lakes, NJ (US)

(21) Appl. No.: 18/724,239

(22) PCT Filed: Dec. 30, 2021
(86) PCT No.: PCT/US2021/073176

§ 371 Date: Jun. 26, 2024

(51) Int. Cl. A61F 2/01; A61F 2/82

(52) U.S. Cl. CPC A61F 2/012; A61F 2/0105; A61F 2/82; A61F 2002/018; A61F 2210/0004; A61F 2220/0016; A61F 2220/0025; A61F 2230/0069

(57) ABSTRACT

A vascular implant including a tubular body of a polymeric material. The body has opposed filter body ends and a longitudinal axis. The body has a generally tubular segmented side wall that includes multiple spaced apart wall panels surrounding a central open-ended bore. The body includes one or more filtering elements in the bore and in between filter ends. One or more filtering elements including a hub at the central longitudinal axis and multiple radially extending filtering arms extend radially between the hub and wall, wherein each arm connects to a wall panel. The tubular body end has anchors that prevent migration and/or tilting.

### CROSS-REFERENCE TO RELATED APPLICATIONS

N/A

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### 1. Field of the Invention

The present invention relates to bioresorbable vascular implants such as filters (e.g., vena cava filters), occlusion devices, stents and convertible stents. More particularly, the present invention relates to an improved vascular implant that bioresorbs into a patient's vascular system (e.g., inferior vena cava, iliofemoral vein, ovarian veins, splenic artery, uterine artery, hepaticartery or other vein/artery vessel). For example, the present invention bioresorbs into a patient's vascular system (e.g. inferior vena cava or iliofemoral vein) after transient risk of pulmonary embolism (PE) has subsided. The entire implant structure could be made of bioresorbable material, such as a bioresorbable polymer so that no implant or implant remnant/element would ultimately be left behind as the entire implant would resorb into vascular tissue. In one or more preferred embodiments, the implant preferably has a body with a tubular outer wall with an interior bore, a multi-panel outer wall, and filtering elements connecting the panels together.

In one or more embodiments, the filter preferably has a hub head that can be annular, ring shaped or rounded, wherein the filtering element connects to the head, annular member or ring shaped hub.

### 2. General Background of the Invention

Vascular implants include various devices that are placed at a selected locale in a patient's blood vessel. One example is a vena cava filter. Other examples include occlusion devices, stents and convertible stents. Various patents have issued for vascular implants. Patents have also issued that relate in general to 3D printing of implants. Examples are listed in the following Table 1. Each patent listed in Table 1 is hereby incorporated herein by reference.

TABLE 1

| PATENT OR | | |
|---|---|---|
| PUBLICATION | | ISSUE DATE |
| NO. | DESCRIPTION | (DD/MM/YYYY) |
| 8,092,484 | EMBOLUS BLOOD CLOT FILTER | 10 Jan. 2012 |
| | WITH POST DELIVERY ACTUATION | |
| 8,092,485 | RECOVERABLE INFERIOR VENA | 10 Jan. 2012 |
| | CAVA FILTER | |
| 8,317,818 | REMOVABLE BLOOD CLOT FILTER | 27 Nov. 2012 |
| | WITH EDGE FOR CUTTING THROUGH | |
| | THE ENDOTHELIUM | |
| 8,420,113 | BIODEGRADABLE MEDICAL | 16 Apr. 2013 |
| | DEVICES WITH ENHANCED | |
| | MECHANICAL STRENGTH AND | |
| | PHARMACOLOGICAL FUNCTIONS | |
| 8,518,072 | JUGULAR FEMORAL VENA CAVA | 27 Aug. 2013 |
| | FILTER SYSTEM | |
| 8,562,638 | EMBOLUS BLOOD CLOT FILTER | 22 Oct. 2013 |
| | WITH FLOATING FILTER BASKET | |
| 8,734,479 | EMBOLUS BLOOD CLOT FILTER | 27 May 2014 |
| | DELIVERY SYSTEM | |
| 8,777,975 | EMBOLUS BLOOD CLOT FILTER | 15 Jul. 2014 |
| | WITH BIO-RESORBABLE COATED | |
| | FILTER MEMBERS | |
| 8,795,351 | MIGRATION RESISTANT EMBOLIC | 5 Aug. 2014 |
| | FILTER | |
| 8,870,943 | STENT STRUCTURE FOR | 28 Oct. 2014 |
| | IMPLANTATBLE MEDICAL DEVICE | |
| 8,992,562 | FILTER DELIVERY SYSTEM | 31 Mar. 2015 |
| 9,220,588 | SYSTEMS, METHODS AND DEVICE | 29 Dec. 2015 |
| | FOR EMBOLIC PROTECTION | |
| 9,393,095 | JUGULAR FEMORAL VENA CAVA | 19 Jul. 2016 |
| | FILTER SYSTEM | |
| 9,421,081 | EMBOLUS BLOOD CLOT FILTER | 23 Aug. 2016 |
| | DELIVERY SYSTEM | |
| 9,445,895 | INTRACARDIAC CAGE AND METHOD | 25 Sep. 2016 |
| | OF DELIVERING SAME | |
| 9,456,888 | REVERSIBLE VASCULAR FILTER | 4 Oct. 2016 |
| | DEVICES AND METHODS | |
| 9,468,513 | EMBOLUS BLOOD CLOT FILTER | 18 Oct. 2016 |
| | WITH BIO-RESORBABLE COATED | |
| | FILTER MEMBERS | |
| 9,561,094 | DEVICES AND METHODS FOR | 7 Feb. 2017 |
| | TREATING VENOUS DISEASES | |
| 9,597,435 | MEDICAL DEVICES HAVING A | 21 Mar. 2017 |
| | BIORESORBABLE COATING LAYER | |
| | WITH A PRE-DETERMINED PATTERN | |
| | FOR FRAGMENTATION | |
| 9,693,851 | FILTER DELIVERY SYSTEM | 14 Jul. 2017 |
| 9,730,781 | EMBOLUS BLOOD CLOT FILTER | 15 Aug. 2017 |
| | REMOVAL SYSTEM AND METHOD | |
| 9,949,816 | IVE FILTER RETRIEVAL SYSTEMS | 24 Apr. 2018 |
| | WITH MULTIPLE CAPTURE MODES | |
| 9,980,804 | VENA CAVA FILTER WITH FILAMENT | 29 May 2018 |
| 10,105,206 | INFERIOR VENA CAVA FILTER WITH | 23 Oct. 2018 |
| | STABILITY FEATURES | |
| 10,188,496 | VENA CAVA FILTER FORMED FROM | 29 Jan. 2019 |
| | A SHEET | |
| 10,188,498 | EMBOLUS BLOOD CLOT FILTER | 29 Jan. 2019 |
| | DELIVERY SYSTEM | |
| 10,226,322 | JUGULAR FEMORAL VENA CAVA | 12 Mar. 2019 |
| | FILTER SYSTEM | |
| 10,258,454 | VISUAL STABILIZER ON ANCHOR | 16 Apr. 2019 |
| | LEGS OF VENA CAVA FILTER | |
| 10,279,078 | CROSSLINKABLE 3D PRINTED | 7 May 2019 |
| | BIOMATERIAL-BASED IMPLANTS | |
| | AND METHODS OF MANUFACTURE | |
| | THEREOF | |
| 10,299,906 | EMBOLUS BLOOD CLOT FILTER | 28 May 2019 |
| | UTILIZABLE WITH SINGLE DELIVERY | |
| | SYSTEM OR A SINGLE RETRIEVAL | |
| | SYSTEN IN ONE OF A FEMORAL OR | |
| | JUGULAR ACCESS | |
| 10,342,654 | IVC FILTER WITH TRANSLATING | 9 Jul. 2019 |
| | HOOKS | |
| 10,368,972 | EMBOLUS BLOOD CLOT FILTER | 6 Aug. 2019 |
| | WITH BIO-RESORBABLE COATED | |
| | FILTER MEMBERS | |
| 10,390,925 | MIGRATION RESISTANT EMBOLIC | 27 Aug. 2019 |
| | FILTER | |
| 10,441,689 | METHODS AND DEVICES FOR THREE- | 15 Oct. 2019 |
| | DIMENSIONAL PRINTING OR | |
| | ADDITIVE MANUFACTURING OF | |
| | BIOACTIVE MEDICAL DEVICES | |
| 10,470,865 | VASCULAR FILTER DEVICE | 12 Nov. 2019 |
| 10,492,898 | EMBOLUS BLOOD CLOT FILTER AND | 3 Dec. 2019 |
| | DELIVERY SYSTEM | |
| 10,512,531 | FILTER DELIVERY SYSTEM | 24 Dec. 2019 |
| 10,531,942 | ABSORBABLE VASCULAR FILTER | 14 Jan. 2020 |
| 10,579,755 | METHOD FOR 3-DAY PRINTING A | 3 Mar. 2020 |
| | CUSTOM BONE GRAFT | |
| 10,624,731 | VASCULAR FILTER SYSTEM | 21 Apr. 2020 |
| 10,729,527 | REMOVABLE EMBOLUS BLOOD | 4 Aug. 2020 |
| | CLOT FILTER | |
| 10,813,738 | TUBULAR FILTER | 27 Oct. 2020 |
| 10,842,608 | VENA CAVA FILTER WITH FILAMENT | 24 Nov. 2020 |
| 2007/064731 | TRANSMISSION APPARATUS WITH | 22 Mar. 2007 |
| | FUNCTION OF MULTI-STEP | |
| | BANDWIDTH ASSIGNMENT TO | |
| | OTHER COMMUNICATION | |
| | APPARATUSES | |
| 2010/0074934 | MEDICAL IMPLANTS WITH A | 25 Mar. 2010 |
| | COMBINATION OF COMPOUNDS | |
| 2016/0166371 | ENDOLUMINAL FILTER DESIGN | 16 Jun. 2016 |
| | VARIATIONS | |
| 2016/0175085 | ENHANCED FLUOROGENIC | 23 Jun. 2016 |
| | ENDOLUMINAL FILTER STRUCTURE | |
| 2017/0105830 | BIODEGRADABLE VASCULAR | 20 Apr. 2017 |
| | FILTER | |
| 2017/0218228 | THREE DIMENSIONAL PRINTING OF | 3 Aug. 2017 |
| | BIO-INK COMPOSITIONS | |
| 2017/0249440 | 3D PRINTING SURGICAL REPAIR | 31 Aug. 2017 |
| | SYSTEMS | |
| 2017/0340429 | VASCULAR FILTER SYSTEM | 30 Nov. 2017 |
| 2018/0168811 | NOVEL BIODEGRADABLE AND NON- | 21 Jun. 2018 |
| | BIODEGRADABLE 3D PRINTED | |
| | IMPLANTS AS A DRUG DELIVERY | |
| | SYSTEM | |
| 2018/0296343 | 3-D PRINTING OF POROUS IMPLANTS | 18 Oct. 2018 |
| 2018/0303616 | 3-D PRINTING OF BONE GRAFTS | 25 Oct. 2018 |
| 2018/0311028 | VENA CAVA FILTER WITH FILAMENT | 1 Nov. 2018 |
| 2019/0110880 | MEDICAL DEVICES AND ANCHORS | 18 Apr. 2020 |
| | THREFOR | |
| 2020/0001540 | ADDITIVE MANUFACTURING ON | 2 Jan. 2020 |
| | UNCONSTRAINED FREEDORM | |
| | SURFACES | |
| 2020/0197150 | VASCULAR FILTER SYSTEM | 25 Jun. 2020 |
| WO2007064731 | HELICAL VENA CAVA FILTER | 7 Jun. 2007 |
| WO2011079287 | REVERSIBLE VASCULAR FILTER | 30 Jun. 2011 |
| | DEVCIES AND METHODS FOR USING | |
| | SAME | |
| WO2016154148 | ARTIFICIAL TYMPANIC MEMBRANE | 29 Sep. 2016 |
| | DEVICES AND USES | |
| WO2018117907 | SHAPE MEMORY POLYMER | 28 Jun. 2018 |
| | COMPOSITE FOR 3D PRINTING OF | |
| | MEDICAL ITEMS | |
| WO2018218085 | THREE-DIMENSIONAL PRINTED | 29 Nov. 2018 |
| | ORGANS, DEVICES, AND MATRICES | |
| WO2019178086 | ELECTROHYDRODYNAMIC | 19 Sep. 2019 |
| | BIOPRINTER SYSTEM AND METHOD | |
| WO2020123945 | FABRIC MATERIAL FOR MEDICAL | 18 Jun. 2020 |
| | DEVICES | |
| EP2363156 | METHOD OF FABRICATING | 7 Sep. 2011 |
| | BIODEGRADABLE MEDICAL | |
| | DEVICES WITH ENHANCED | |
| | MECHANICAL STRENGTH AND | |
| | PHARMACOLGOICAL FUNCTIONS | |

### BRIEF SUMMARY OF THE INVENTION

The present invention provides a vascular implant preferably having a tubular body of a polymeric material, having opposed filter body ends and a central longitudinal axis.

In one or more preferred embodiments, the body has a generally tubular segmented side wall that preferably includes multiple spaced apart wall panels surrounding a central open ended bore. The segmented tubular wall allows the device to be compressed to fit inside a delivery system and reduces the amount of material that needs to be resorbed.

In one or more embodiments, the body preferably includes one or more filtering elements in said bore and in between filter body ends.

In one or more embodiments, one or more filtering elements preferably include a hub at the central longitudinal axis and multiple radially extending filtering arms that extend between the hub and the wall, wherein each arm connects to the hub and a wall panel.

In one or more embodiments, the tubular body can have anchors that prevent migration and/or tilting.

In one or more embodiments, the body is of a resorbable material.

In one or more embodiments, the body is of a material that is too soft to be machined.

In one or more embodiments, the opposed ends of the body preferably include an upper end and a lower end.

In one or more embodiments, one or more of the arms extend outwardly from the hub toward one of said ends.

In one or more embodiments, one or more of said arms extend from the hub upwardly toward the body upper end.

In one or more embodiments, the body is of a polymeric material that has a durometer reading that is between 10 Shore A and 75 Shore D.

In one or more embodiments, the body is of a polymeric material that has a durometer reading that is between 35 Shore A and 75 Shore D.

In one or more embodiments, the body is of a polymeric material that has a durometer reading that is between 10 Shore A and 40 Shore A.

In one or more embodiments, the body and one or more filtering elements define a vena cava filter.

In one or more embodiments, the body and one or more filtering elements define an occlusion device.

In one or more embodiments, the body and one or more filtering elements define a convertible stent.

In one or more embodiments, the tubular body is of a polymeric material, has opposed filter body ends and a central longitudinal axis.

In one or more embodiments, the body preferably has a segmented side wall that includes multiple circumferentially spaced apart wall panels and a central bore. The body preferably includes one or more filtering elements in said bore and in between filter body ends.

In one or more embodiments, one or more filtering elements include a hub at a central longitudinal axis and multiple radially extending filtering arms that extend radially between the hub and the wall, wherein each arm connects to the hub and a wall panel.

In one or more embodiments, the tubular body preferably has anchors that prevent migration and/or tilting.

In one or more embodiments, the body and one or more filtering elements define a stent.

In one or more embodiments, each filtering arm preferably forms an acute angle with the central longitudinal axis.

In one or more embodiments, each filtering member/connecting member/strut/arm/appendage is connected to head/ring/annular member/hub via holes/sockets/bores and features a ball and socket connection.

In one or more embodiments, the holes through which each filtering member pass can be conically or frusto-conically shaped.

In one or more embodiments, each filtering member/connecting member/strut/arm/appendage has a ball and socket connection to both the central head/ring/annular member/hub and each outer wall section/panel.

In one or more embodiments, each outer wall section has a hole or socket or bore for a filter member to pass through.

In one or more embodiments, each hole or socket or bore on the outer wall can be open ended and can be conically or frusto-conically shaped in the same manner as the annular ring.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a top view of a preferred embodiment of the apparatus of the present invention;

FIG. 2 is a side, elevation view of a preferred embodiment of the apparatus of the present invention;

FIG. 3 is a perspective view of a preferred embodiment of the apparatus of the present invention;

FIG. 4 is a perspective view of a preferred embodiment of the apparatus of the present invention;

FIG. 5 is a top view of a preferred embodiment of the apparatus of the present invention;

FIG. 6 is a close up, perspective view showing the connection of the head, hub, ring or annular member with a filtering member, connecting member, strut, arm or appendage;

FIG. 7 is perspective view of another preferred embodiment of the present invention; and

FIG. 8 is a top view of the preferred embodiment of FIG. 7.

### DETAILED DESCRIPTION OF THE INVENTION

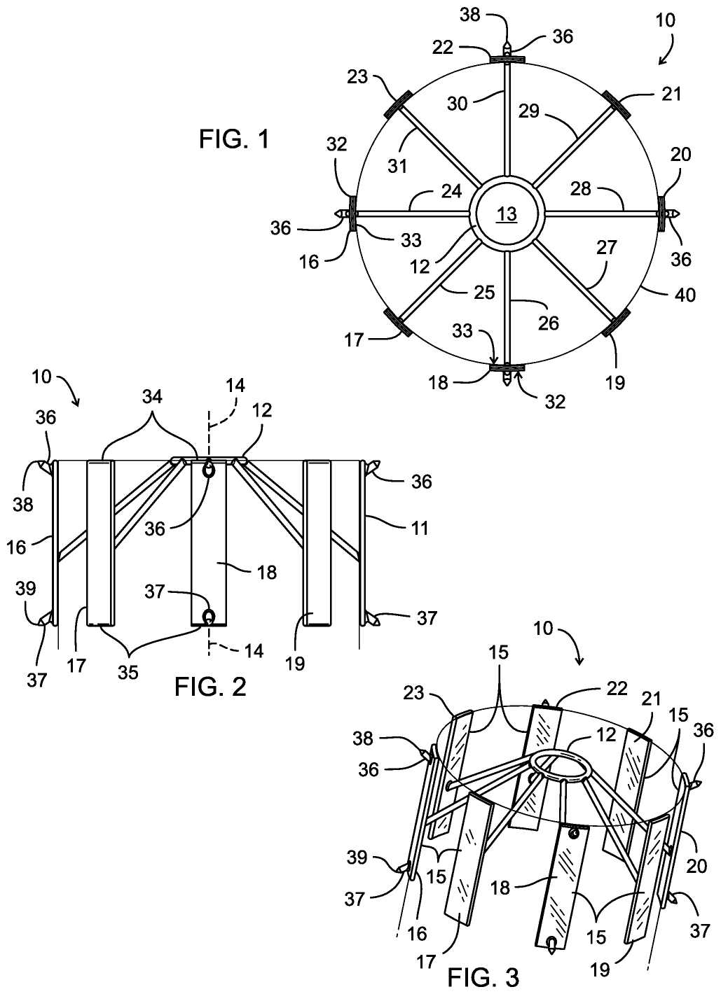

FIGS. 1-3 show a preferred embodiment of the apparatus of the present invention designated generally by the numeral 10. Vascular implant 10 includes an implant body 11 that can be of a polymeric material or polymeric resorbable material such as a bioresorbable polymer material. In this embodiment, the body 11 has a tubular shape with opposed filter body ends 34, 35 and a central longitudinal axis 14. Body 11 can be of a material that is too soft to be machined such as a durometer of below 20 Shore D. Body 11 can be of a material that has a durometer reading of between 10 Shore A and 40 Shore A. Body 11 has a head, hub, ring or annular member 12 with central opening or aperture 13. Implant body 11 has a central longitudinal axis 14 that extends generally through the center of opening 13 (see FIG. 2). Head 12 could be of another shape such as circular, oval, rounded, polygonal or other shape that enables attachment of filter legs thereto.

Body 11 has a segmented side or outer wall 15 comprised of a plurality of circumferentially spaced apart wall sections or panels 16-23. The wall sections or panels 16-23 are distributed around the central longitudinal axis. The wall sections or panels 16-23 extend in this embodiment parallel to the central longitudinal axis. When placed in a vessel, the outwards facing surface of the wall sections or panels 16-23 abut to the vessel and extend in a direction more or less parallel to the direction of blood-flow through the vessel. Due to the wall sections or panels 16-23, the implant can reduce the risk of damage to the vessel. In case the wall sections or panels 16-23 are of a (bio-)resorbable material, they will be resorbed into the vascular tissue after a certain period of time. Panels or wall sections 16-23 can be generally rectangular in shape. Panels or wall sections 16-23 could be other shapes such as square, oval, rectangular, or provided with chamfer or fillets on corners of each of the square or rectangular shapes.

Each panel or wall section 16-23 can have a convex outer surface 32 and a concave inner surface 33, as seen in FIG. 1. The concave surfaces 33 can track a circular path or circle 40 as seen in FIG. 1. Path 40 can also be an oval shape. Other shapes for outer wall 15 can be square or oval. The inner surfaces 33 form a central bore.

The panels 16-23 define a central bore in which one or more filtering elements extend. More specifically, these filtering elements extend in radial direction from the center of the tubular body 11 up to a respective wall section or panel 16-23 and lay in axial direction between the filter body ends 34,35. Each wall section or panel 16-23 is connected to head, hub, ring or annular member 12 with a filtering member, connecting member, strut, arm or appendage 24, 25, 26, 27, 28, 29, 30 or 31 as seen in FIGS. 1-3. The head 12 can thus be seen as a hub at the central longitudinal axis, and the filtering members, connecting members, struts, arms or appendages 24-31 as multiple radially extending filtering arms that extend radially between the hub and the segmented wall, in this embodiment from the hub up to the segmented wall. These members 24-31 act as filters that trap emboli at the downstream side of the filters, within the central bore illustrated with circle 40 in FIG. 1. Emboli can also be trapped by annular member, ring, hub, or head 12.

As is apparent from FIGS. 2-3 for example, the members 24-31 may extend from the respective wall section or panel 16-23 upstream, in a direction of blood flow, and radially inwards to the hub 12. Thereby, the members 24-31 form a dome-shaped filter in the bore that when placed in the vessel effectively retains blood-cloths. Head, hub, ring or annular member 12 can be located closer to the first end portion 34 than to the second end portion 35, and in this embodiment is located, in the axial direction at the first end portion 34. This provides for a large volume to retain blood clots, when the implant 10 is placed in the vessel and the blood flows in the direction from second end portion 35 to first end portion 34 and reduces the risks that the flow of blood through the filter becomes obstructed.

In FIGS. 1-3, hub, head or annular member 12 connects to panel or wall section 16 with strut, filtering member, arm or appendage 24. Similarly, appendage 25 connects panel 17 to hub or ring 12. Panel 18 connects to hub or ring 12 with member 26. Panel 19 connects to hub or ring 12 with member 27. In this fashion, panels 20, 21, 22 and 23 connect to ring 12 with members 28, 29, 30, 31 respectively.

One or more of the panels or wall sections 16-23 is preferably provided with one or more anchors. In FIGS. 1-3, panels 16, 18, 20 and 22 are each fitted or equipped with two (2) anchors 36, 37. Filter body 11 has first end portion 34 and second end portion 35 (see FIG. 2). Each anchor 36 extends toward first end 34 as seen in FIG. 2. Each anchor 36 can form an acute angle with axis 14. Each anchor 37 can extend toward second end 35. Each anchor 37 can form an acute angle with axis 14. Each anchor can be provided with a sharp tip. Each anchor 36 has sharp point or tip 38. Each anchor 37 has sharp point or tip 39 (see FIG. 3).

Once implemented at a selected locale in a patient's vascular system (e.g., using a catheter), the anchors 36, 37 prevent migration and/or tilting of the filter body 11 in either direction (either toward end 34 or toward end 35). When of a bioresorbable material, the bioresorption starts as soon as the implant is exposed to the blood and like many of the standard polymers degrades over time via polymer breakdown and absorption of the byproducts. This bioresorption can be tuned as needed to ensure the implant maintains structural filtering integrity until transient risk of pulmonary embolism has subsided. As noted, the segmented tubular wall reduces the amount of material that needs to be resorbed.

The implant 10 could be deployed with either a femoral or jugular approach. Such a deployment could employ a pusher or pusher apparatus/mechanism such as one specified in one or more of the patents listed in Table 1. An example is U.S. Pat. No. 8,518,072 naming Jonathan Miller as inventor and assigned to C.R. Bard, Inc. Implant 10 could also be a balloon-mounted implant that is then expanded with balloon dilation, as seen, for example, in balloon expandable stents.

Filter body can be manufactured with 3D printing, such as a one piece polymeric body 11. For inferior vena cava pulmonary embolism prevention, the diameter of implant 10 can be between about 15-30 mm. For iliofemoral pulmonary embolism protection, the diameter of implant 10 can be between about 6-20 mm. For other smaller vessels for occlusion, implant 10 can have a diameter of between about 2-8 mm. The length of implant 10 can be between about 5-50 mm.

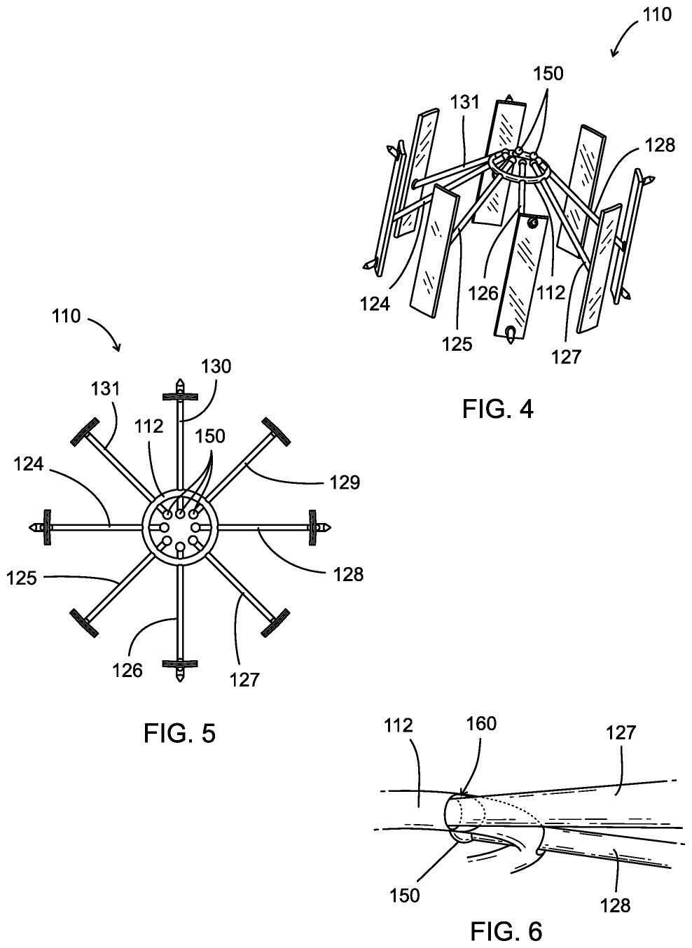

FIGS. 4-6 show another embodiment of the present invention designated generally by the numeral 110. Vascular implant 110 in FIGS. 4-5 is similar to vascular implant 10 shown in FIGS. 1-3 and except as noted, contains the same parts. In vascular implant 110, each filtering member/connecting member/strut/arm/appendage 124, 125, 126, 127, 128, 129, 130, and 131 is connected to head/ring/annular member/hub 112 via holes/sockets/bores 160 and features a ball 150 and socket 160 connection. The end of each filtering member/connecting member/strut/arm/appendage 124, 125, 126, 127, 128, 129, 130, and 131 contains ball 150 which prevents each filtering member from fully separating from hub 112. The hole/socket 160 through which each filtering member passes can be conically or frusto-conically shaped, as depicted in FIG. 6, to allow for a change of angle of each filtering member 124-131 relative to hub 112 and accordingly, each wall section/panel 16-23. Hole/socket 160 is open ended and thus allows for each section/panel 16-23 to be pulled inward toward the annular ring 112, for example, when collapsing the vascular implant 110. The ball and socket connection or feature as shown in FIGS. 4-6 is preferably applicable when the body of the vascular implant is on the lower end of the durometer range, for example when the material has a hardness that is within a durometer reading of between about 10 Shore A and 40 Shore A.

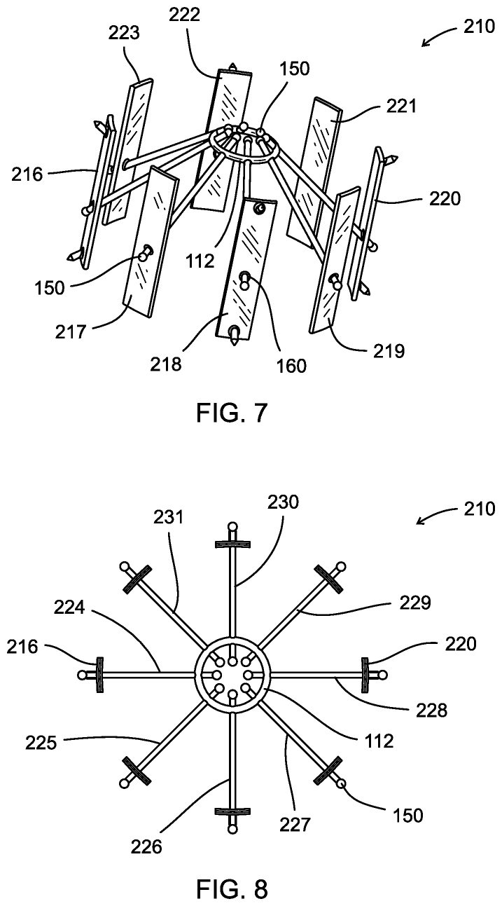

In another embodiment, shown in FIGS. 7 and 8, each filtering member/connecting member/strut/arm/appendage 224-231 has a ball 150 and socket 160 connection to both the central head/ring/annular member/hub 212 and each outer wall section/panel 216-223. Vascular implant 210 is otherwise similar to vascular implant 110. Each outer wall section/panel 216-223 of vascular implant 223 has a hole or socket or bore 160 for the filtering member to pass through. Each hole or socket or bore 160 on the outer wall is open ended and can be conically or frusto-conically shaped in the same manner as annular ring 112. The vascular implant of FIGS. 7 and 8 can be of a material that has a hardness of between 10 Shore A and 75 Shore D. When the material is on the higher end of the durometer range, for example when the material has a hardness that is within a durometer reading of between about 65 Shore A and 75 Shore D, a vascular implant with ball and socket features at both the central hub and outer walls, such as vascular implant 210 of FIGS. 7 and 8, is preferred. Any embodiment can be utilized when the vascular implant material is on the lower end of the durometer range, for example, between 10 Shore A and 40 Shore A. In FIGS. 7 and 8, the wall panels 216-223, when deployed, can move radially from hub 112 so as to engage with a vessel wall.

Radial movement can be effected by balloon deployment, wedge, or inherent spring force (for example, if the filaments were attached but of sufficient elasticity to deform without breaking). Preferably, wall panels 216-223 when deployed move axially rather than radially when going from a transported to expanded configuration. Although struts 124-131 of FIGS. 4-5 and struts 224-231 of FIGS. 7-8 are shown extending through hub 112, hub 112 could alternatively be shaped so as to allow for angular motion only and not axial, of the struts. The embodiments shown which allow angular and axial motion would allow greater mobility. In FIGS. 7 and 8, balls 150 are shown extending outside of the plane of wall panels 216-223 in order to demonstrate that struts 224-231 can pass through the wall panels 216-223. However, when deployed and engaged with a vessel wall, the balls 150 for each strut 224-231 preferably are housed within sockets 160 of wall panels 216-225 so as to allow greater engagement of the wall panels 216, 218, 220, 222 with the vessel wall.

The vascular implant can thus be characterized by comprising:
a) a tubular body of a polymeric material, comprising opposed filter body ends and a central longitudinal axis;
b) said body comprising a segmented side wall that includes multiple spaced apart wall panels and that defines a central bore;
c) said body comprising one or more filtering elements in said bore and in between filter body ends;
d) said one or more filtering elements comprising a hub at said central longitudinal axis and multiple radially extending filtering arms that extend radially between said hub and said wall, wherein each arm connects to said hub and a said wall panel;
e) said tubular body comprising anchors that prevent migration and/or tilting of the implant.

Without limitation, the implant may further be characterized by one or more of the following statements.

Statement 1: A vascular implant, comprising:
a) a tubular body of a polymeric material, comprising opposed filter body ends and a central longitudinal axis;
b) said body comprising a generally tubular segmented side wall that includes multiple spaced apart wall panels surrounding a central open ended bore;
c) said body comprising one or more filtering elements in said bore and in between filter body ends;
d) said one or more filtering elements comprising a hub at said central longitudinal axis and multiple radially extending filtering arms that extend radially between said hub and said wall, wherein each arm connects to said hub and a said wall panel; and
e) said tubular body comprising anchors that prevent migration and/or tilting.

Statement 2: The vascular implant of statement 1 wherein the body is of a resorbable material.

Statement 3: The vascular implant of one or more of the preceding statements wherein the body is of a material that is too soft to be machined.

Statement 4: The vascular implant of one or more of the preceding statements wherein said opposed ends include an upper end and a lower end.

5: The vascular implant of one or more of the preceding statements wherein one or more of said arms extend from said hub toward one of said ends.

Statement 6: The vascular implant of statement 4 wherein one or more of said arms extend from said hub upwardly toward said upper end.

Statement 7: The vascular implant of one or more of the preceding statements wherein said polymeric material has a hardness that is a durometer reading of between about 35 Shore A and 75 Shore D.

Statement 8: The vascular implant of one or more of the preceding statements wherein said polymeric material is too soft to be machined.

Statement 9: The vascular implant of one or more of the preceding statements wherein the body and one or more filtering elements define a vena cava filter.

Statement 10: The vascular implant of one or more of the preceding statements wherein the body and one or more filtering elements define an occlusion device.

Statement 11: The vascular implant of one or more of the preceding statements wherein the body and one or more filtering elements define a convertible stent.

Statement 12: A vascular implant, comprising:
a) a tubular body of a polymeric material, comprising opposed filter body ends and a central longitudinal axis;
b) said body comprising segmented side wall that includes multiple circumferentially spaced apart wall panels and a central bore;
c) said body comprising one or more filtering elements in said bore and in between filter body ends;
d) said one or more filtering elements comprising a hub at said central longitudinal axis and multiple radially extending filtering arms that extend radially between said hub and said wall, wherein each arm connects to said hub and a said wall panel; and
e) said tubular body comprising anchors that prevent migration and/or tilting;

Statement 13: The vascular implant of statements 12 wherein the body is of a resorbable material.

Statement 14: The vascular implant of one or more of statements 12-13, wherein the body is of a material that is too soft to be machined.

Statement 15: The vascular implant of one or more of statements 12-14, wherein said polymeric material is too soft to be machined.

Statement 16: The vascular implant of one or more of statements 12-15, wherein the body and one or more filtering elements define a vena cava filter.

Statement 17: The vascular implant of one or more of statements 12-16, wherein the body and one or more filtering elements define an occlusion device.

Statement 18: The vascular implant of one or more of statements 12-17, wherein the body and one or more filtering elements define a convertible stent.

Statement 19: The vascular implant of one or more of statements 12-18, wherein each filtering arm forms an acute angle with said central longitudinal axis.

Statement 20: A vascular implant, comprising:
a) a tubular body of a polymeric material, comprising opposed filter body ends and a central longitudinal axis;
b) said body comprising a generally tubular segmented side wall that includes multiple spaced apart wall panels surrounding a central open ended bore;
c) said body comprising one or more filtering elements in said bore and in between filter body ends;
d) said one or more filtering elements comprising a hub at said central longitudinal axis and multiple radially extending filtering arms that extend radially between said hub and said wall, wherein each arm connects to said hub and a said wall panel;
e) wherein said arms are spaced circumferentially apart; and
f) said tubular body comprising anchors that prevent migration and/or tilting.

The following is a list of parts and materials suitable for use in the present invention.

PARTS LIST

| Part Number | Description |
|---|---|
| 10 | vascular implant |
| 11 | implant body/filter body |
| 12 | head/ring/annular member/hub |
| 13 | opening/aperture |
| 14 | central longitudinal axis |
| 15 | segmented wall/outer wall |
| 16 | wall section/panel |
| 17 | wall section/panel |
| 18 | wall section/panel |
| 19 | wall section/panel |
| 20 | wall section/panel |
| 21 | wall section/panel |
| 22 | wall section/panel |
| 23 | wall section/panel |
| 24 | filtering member/connecting |
| | member/strut/arm/appendage |
| 25 | filtering member/connecting |
| | member/strut/arm/appendage |
| 26 | filtering member/connecting |
| | member/strut/arm/appendage |
| 27 | filtering member/connecting |
| | member/strut/arm/appendage |
| 28 | filtering member/connecting |
| | member/strut/arm/appendage |
| 29 | filtering member/connecting |
| | member/strut/arm/appendage |
| 30 | filtering member/connecting |
| | member/strut/arm/appendage |
| 31 | filtering member/connecting |
| | member/strut/arm/appendage |
| 32 | convex outer surface |
| 33 | concave inner surface |
| 34 | first end portion |
| 35 | second end portion |
| 36 | anchor |
| 37 | anchor |
| 38 | sharp point/tip |
| 39 | sharp point/tip |
| 40 | circular path/circle |
| 110 | vascular implant |
| 112 | head/ring/annular member/hub |
| 124 | filtering member/connecting |
| | member/strut/arm/appendage |
| 125 | filtering member/connecting |
| | member/strut/arm/appendage |
| 126 | filtering member/connecting |
| | member/strut/arm/appendage |
| 127 | filtering member/connecting |
| | member/strut/arm/appendage |
| 128 | filtering member/connecting |
| | member/strut/arm/appendage |
| 129 | filtering member/connecting |
| | member/strut/arm/appendage |
| 130 | filtering member/connecting |
| | member/strut/arm/appendage |
| 131 | filtering member/connecting |
| | member/strut/arm/appendage |
| 150 | rounded end/ball |
| 160 | hole/socket/conically shaped |
| | opening/bore |
| 210 | vascular implant |
| 216 | wall section/panel |
| 217 | wall section/panel |
| 218 | wall section/panel |
| 219 | wall section/panel |
| 220 | wall section/panel |
| 221 | wall section/panel |
| 222 | wall section/panel |
| 223 | wall section/panel |
| 224 | filtering member/connecting |
| | member/strut/arm/appendage |
| 225 | filtering member/connecting |
| | member/strut/arm/appendage |
| 226 | filtering member/connecting |
| | member/strut/arm/appendage |
| 227 | filtering member/connecting |
| | member/strut/arm/appendage |
| 228 | filtering member/connecting |
| | member/strut/arm/appendage |
| 229 | filtering member/connecting |
| | member/strut/arm/appendage |
| 230 | filtering member/connecting |
| | member/strut/arm/appendage |
| 231 | filtering member/connecting |
| | member/strut/arm/appendage |

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A vascular implant, comprising:
a) a tubular body of a polymeric material, having opposed filter body ends and a central longitudinal axis;
b) said body having a generally tubular segmented side wall that includes multiple spaced apart wall panels surrounding a central open ended bore;
c) said body including one or more filtering elements in said bore and in between filter body ends;
d) said one or more filtering elements including a hub at said central longitudinal axis and multiple radially extending filtering arms that extend radially between said hub and said wall, wherein each arm connects to said hub and a said wall panel; and
e) said tubular body having anchors that prevent migration and/or tilting.

2. The vascular implant of claim 1 wherein the body is of a resorbable material.

3. The vascular implant of claim 1 wherein the body is of a material that is too soft to be machined.

4. The vascular implant of claim 1 wherein said opposed ends include an upper end and a lower end.

5. The vascular implant of claim 1 wherein one or more of said arms extend from said hub toward one of said ends.

6. The vascular implant of claim 4 wherein one or more of said arms extend from said hub upwardly toward said upper end.

7. The vascular implant of claim 1 wherein said polymeric material has a hardness that is a durometer reading of between about 35 Shore A and 75 Shore D.

8. The vascular implant of claim 1 wherein said polymeric material is too soft to be machined.

9. The vascular implant of claim 1 wherein the body and one or more filtering elements define a vena cava filter.

10. The vascular implant of claim 1 wherein the body and one or more filtering elements define an occlusion device.

11. The vascular implant of claim 1 wherein the body and one or more filtering elements define a convertible stent.

12. A vascular implant, comprising:
a) a tubular body of a polymeric material, having opposed filter body ends and a central longitudinal axis;
b) said body having segmented side wall that includes multiple circumferentially spaced apart wall panels and a central bore;
c) said body including one or more filtering elements in said bore and in between filter body ends;
d) said one or more filtering elements including a hub at said central longitudinal axis and multiple radially extending filtering arms that extend radially between said hub and said wall, wherein each arm connects to said hub and a said wall panel; and
e) said tubular body having anchors that prevent migration and/or tilting;

13. The vascular implant of claim 12 wherein the body is of a resorbable material.

14. The vascular implant of claim 12 wherein the body is of a material that is too soft to be machined.

15. The vascular implant of claim 12 wherein said polymeric material is too soft to be machined.

16. The vascular implant of claim 12 wherein the body and one or more filtering elements define a vena cava filter.

17. The vascular implant of claim 12 wherein the body and one or more filtering elements define an occlusion device.

18. The vascular implant of claim 12 wherein the body and one or more filtering elements define a convertible stent.

19. The vascular implant of claim 12 wherein each filtering arm forms an acute angle with said central longitudinal axis.

20. A vascular implant, comprising:
a) a tubular body of a polymeric material, having opposed filter body ends and a central longitudinal axis;
b) said body having a generally tubular segmented side wall that includes multiple spaced apart wall panels surrounding a central open ended bore;
c) said body including one or more filtering elements in said bore and in between filter body ends;
d) said one or more filtering elements including a hub at said central longitudinal axis and multiple radially extending filtering arms that extend radially between said hub and said wall, wherein each arm connects to said hub and a said wall panel;
e) wherein said arms are spaced circumferentially apart; and
f) said tubular body having anchors that prevent migration and/or tilting.

21. The inventions substantially as shown and/or described herein.
